(19) United States
(12) Patent Application Publication
(10) Pub. No.: US 2003/0059370 A1
(43) Pub. Date: Mar. 27, 2003

(54) In vivo diagnostic apparatus and methods thereof

(76) Inventors: Rodolfo C. Quijano, Laguna Hills, CA (US); Hosheng Tu, Newport Coast, CA (US)

Correspondence Address: Hosheng Tu, 15 Riez, Newport Coast, CA, 92657, US

(21) Appl. No.: 09/963,189

(22) Filed: Sep. 25, 2001

(51) Int. Cl. A61K 51/00; A61K 49/00

(52) U.S. Cl. 424/1.49; 424/9.1

(57) ABSTRACT

A method for diagnosing an antigen in vivo comprising introducing a diagnosing element to contact with a biological fluid containing said antigen at a site inside a patient, wherein said diagnosing element comprises antibody conjugate-able to said antigen adapted to form antigen-antibody conjugate; quantifying the antigen-antibody conjugate, wherein said quantification of the antigen-antibody conjugate is performed by means for assaying said antigen-antibody conjugate in vivo, and/or prorating a quantity of antigen-antibody conjugate to a reference quantity of antigen.

### FIELD OF THE INVENTION

The present invention relates to an in vivo detection/measuring apparatus and methods for diagnosis, detection and identification of the markers and their sources in a patient.

### BACKGROUND OF THE INVENTION

Cardiovascular diseases are the single most common cause of morbidity and mortality in the developed world. Several risk factors for cardiovascular disorders have been described and in wide clinical use in the detection of individuals at high risk. Such screening tests include evaluations of total cholesterol level, HDL cholesterol level, ratio of cholesterol levels, C-reactive protein (CRP), and the like. Accumulating data suggests that the current in vitro diagnostic tests to determine whether certain therapies can be expected to be more or less effective are insufficient in terms of specificity and real-time diagnosis.

It is recognized that inflammation and embolization in ischemic heart diseases may be associated with atherosclerotic coronary artery plaque fissure, rupture, or erosion. An acute ischemic heart disease associated with an inflamed artery may include the evidence or markers, such as CRP, tissue-type plasminogen activator (t-PA) antigen, fibrinogen, interleukin-6 (IL-6), or the like diagnosed in vitro (Topol, J Invas Cardiol 2000;12:2B-7B). Ridkler and associates (U.S. Pat. No. 6,040,147) have advanced the field by showing that CRP is a major independent risk marker for atherosclerotic disease adverse outcomes. It is suggested that CRP is as important as the ratio of total cholesterol to high-density lipoprotein (HDL) and that the two key markers are independent and additive for predicting prognosis. Kudsk in U.S. Pat. No. 5,882,872 discloses use of an IL-6 assay for predicting the development of post-trauma complications. It is one object of the present invention to detect the markers, including CRP, tissue-type plasminogen activator (t-PA) antigen, fibrinogen, interleukin-6 (IL-6), or the like, associated with vulnerable plaque, trauma or ischemic heart disease in vivo leading to appropriate therapeutic treatments.

Apoptosis is generally referred as programmed cell death. In the course of evolution a number of mechanisms, such as DNA repair, production of stress proteins, antioxidant defense systems, and poly(ADP-ribosyl) polymerase activation, have emerged that allow the cell to cope with a variety of potentially harmful agents. A failure of these mechanisms and/or deprivation of growth factors seem able to induce an active process of programmed cell death characterized by endonuclease activity and DNA fragmentation. Apoptosis is a clean and physiological way to get rid of cells and keep the homeostasis of cell population and is usually diagnosed in vitro. It is another object of the present invention to detect the markers associated with apoptosis in ischemic heart diseases in vivo leading to appropriate therapeutic treatments.

Various studies have demonstrated that circulating B-type natriuretic peptide (BNP) concentrations increase with the severity of congestive heart failure (CHF) in in vitro studies. BNP concentrations are normally much lower than ANP concentrations, but as the severity of CHF advances, plasma BNP increases progressively more than respective ANP values. Therefore, BNP appears to be a more useful marker to distinguish between normal subjects and patients in the early stages of CHF. It is still another object of the present invention to detect the BNP markers associated with congestive heart failures in vivo leading to appropriate therapeutic treatments.

The invention is to provide an in vivo diagnostic apparatus and methods for diagnosing, detecting and identifying the disease markers and/or their sources in a patient with respect to site specificity and sensitivity. Additionally, the present invention is to provide a point-of-detection analysis on an essentially real-time or continuous monitoring of the markers inside a patient leading to effective therapies.

### SUMMARY OF THE INVENTION AND DESCRIPTION

It is one object of the present invention to provide an immunoassay system for determining the presence or amount of a cardiovascular marker or a group of cardiovascular markers in a whole blood, plasma or serum site-specifically inside a patient suspected of containing said markers from damaged heart muscle or blood vessels. The system may comprise: a) an antibody coupled or immobilized on a diagnosing element to be inserted into the body of a patient, said antibody capable of specifically binding an antigen-type marker to form an antigen-antibody conjugate; b) formation of a reaction mixture, for example, an antigen-antibody conjugate, involving the whole blood, plasma or serum of a patient in vivo; c) performing assay for the antigen-antibody conjugate in vivo, whereby the antigen-antibody conjugate produces a detectable signal; and, d) relation of detectable signal to the presence or amount of said marker from reference.

It is another object to provide a method for diagnosing an antigen in vivo comprises introducing a diagnosing element to contact with a biological fluid containing said antigen at a site inside a patient, wherein said diagnosing element comprises antibody conjugate-able to said antigen adapted to form antigen-antibody conjugate; and quantifying the antigen-antibody conjugate, wherein said quantification of the antigen-antibody conjugate is performed by means for assaying the antigen-antibody conjugate in vivo. The method may further comprise a step of prorating a quantity of antigen-antibody conjugate to a reference quantity of antigen.

The method comprises means for assaying the antigen-antibody conjugate by either an immunoradiometric assay or a radioimmunoassay. The quantification of the antigen-antibody conjugate may be performed by means for assaying the antigen-antibody conjugate in vivo by impedance detection means for measuring an impedance between two spaced-apart metal contact-electrodes mounted on the diagnosing element, the antibody being immobilized and coupled to both ends of said metal contact-electrodes that are mounted on the diagnosing element at about a tip portion of a catheter adapted for percutaneously inserting into a body conduit of a patient. The antigen of interest in the present invention may comprise C-reactive protein, B-type natriuretic peptide, interlukin-6, tissue-type plasminogen activator (t-PA) antigen, and the like.

The diagnosing element for antigen assay in vivo may be mounted at about a tip portion of a catheter adapted for percutaneously inserting into a body conduit of a patient. It is also applicable to be mounted at a tip portion of a trocar, a handpiece, a probe, an optic fiber, an endoscopic instrument, a guidewire, a cannula, and other suitable insertable biopsies devices or forceps.

The biological fluid may be selected from the group consisting of whole blood, plasma and serum. Further, the biological fluid may also be selected from the group consisting of cerebrospinal fluid, peritoneal fluid, pleural fluid, lymphatic fluid, and joint fluid.

It is still another object to provide a method for assessing vulnerability of a patient in vivo having vulnerable plaque comprising introducing a diagnosing element to contact with a body fluid of the patient at a site inside the patient, the body fluid containing a substance associated with the vulnerable plaque, wherein said diagnosing element comprises a receptor protein conjugate-able to said substance; quantifying the receptor protein that conjugate-ably reacts with said substance; and comparing a level of the reacted substance of the patient to a reference data pool from healthy patients.

The method for quantifying the receptor protein that conjugate-ably reacts with said substance may comprise radioimmunoassay, an agglutination procedure or a nephelometry procedure. The substance associated with the vulnerable plaque that is conjugate-able to said receptor protein may be selected from a group consisting of C-reactive protein, interlukin-6, B-type natriuretic peptide, and tissue-type plasminogen activator (t-PA) antigen.

In general, it is also an object of the present invention to provide a method for assessing an individual's risk profile of developing a future cardiovascular disorder with atherosclerotic disease. The method may comprise obtaining at least a level of a marker of inflammation about a region of atherosclerotic disease in vivo in the individual; comparing the at least a level of the marker with levels from various sites of said region of the atherosclerotic disease; and identifying a site inside the individual having the level of the marker sufficiently higher than the remaining levels adapted for therapeutic treatment.

### BRIEF DESCRIPTION OF THE DRAWINGS

Additional objects and features of the present invention will become more apparent and the invention itself will be best understood from the following Detailed Description of Exemplary Embodiments, when read with reference to the accompanying drawings.

FIG. 1 is a schematic diagram showing a method for diagnosing an antigen in vivo comprising a diagnosing element having antibody conjugate-able to the antigen adapted to form antigen-antibody conjugate for assay according to the principles of the present invention.

FIG. 2 is a medical apparatus having a diagnosing element mounted at about a tip portion of the apparatus adapted for percutaneously inserting into a body conduit of a patient for antigen assay.

FIG. 3 is one embodiment of the tip section of the medical apparatus of FIG. 2, having the capability of diagnosing an antigen in a patient in vivo.

FIG. 4 is one preferred embodiment of the tip section of the medical apparatus of FIG. 2, having the capability of diagnosing an antigen in a patient in vivo.

### DETAILED DESCRIPTION OF EXEMPLARY EMBODIMENTS

The following detailed description is of the best presently contemplated modes of carrying out the invention. This description is not to be taken in a limiting sense, but is made merely for the purpose of illustrating general principles of embodiments of the invention. The scope of the invention may be best defined by the appended claims.

The present invention relates to an in vivo detection apparatus and methods for diagnosis, detection and identification of the source of markers in a patient, particularly those markers associated with the diseased, impaired, degenerative, or dysfunctional organs/tissue. It is one embodiment for the present invention to provide an apparatus for point-of-detection to locate the source of markers site-specifically in vivo.

Antigen and Antibody

Antigen is a substance that evokes the production of an antibody when introduced into the body of an animal. Antigens can enter the body through the respiratory tract, digestive tract, skin, or blood vessels. The most common antigens are proteins such as those found in bacteria and virus.

Antibody is a normally occurring protein molecule that is produced in the body of cells called lymphocytes and that acts primarily as a defense against invasion by foreign substances. An “antibody” or “receptor protein” refers herein to: a monoclonal antibody, a polyclonal antibody, a binding fragment of an antibody, a recombinant antibody, or a receptor protein that specifically binds to a target. An important component of the immune system, antibodies are found in the blood of all vertebrates, in the fraction of the blood called gamma globulin (γ-globulin). The binding of antibodies to the surfaces of antigens can neutralize and eliminate the harmful antigens in any or all of three ways: (1) by directly inactivating them; (2) by enabling other blood cells to engulf and destroy them (e.g. phagocytosis); (3) by weakening their surfaces and rendering them vulnerable to destruction by other blood proteins.

The five known classes of antibody are distinguished by the letters M, G, E, A, and D; all are preceded by the abbreviation Ig for immunoglobulin, another name for antibody. IgG is the predominant antibody in serum. In particular, a sensitive antibody which is useful in an immunoassay distinguishes one form of antigen from another form and an insensitive antibody which is useful in an immunoassay does not distinguish one form of antigen from another. The sensitivity or insensitivity of an antibody is exhibited in an immunoassay. To determine whether an antibody is sensitive or insensitive, the antibody is tested with each antigen form independently to yield the assay response of the antibody for each antigen form.

C-reactive protein (CRP) is a pentameric globulin with mobility near the gamma zone. It rises rapidly, but nonspecifically in response to tissue injury and inflammation. It is particularly useful in detection of occult infections, acute appendicitis, particularly in leukemia and in post-operative patients. In uncomplicated postoperative recovery, CRP peaks on the 3^{rd} postop day and returns to preop levels by day 7. It may also be helpful in evaluation of extension or reinfarction after myocardial infarction and in following response to therapy in rheumatic disorders.

Immunoassay

A preferred immunoassay for C-reactive protein, B-type natriuretic peptide, interlukin-6, or other antigens involves conjugation of an antibody or a cocktail of antibodies to one of the aforementioned substance to form an antigen-antibody conjugate, which is capable of being assayed using immunological methods. One skilled in the art will recognize that such an antigen-antibody conjugate is routinely performed on an in vitro basis. Principles of radioimmunotherapy for hematologies and oncologists show that radiolabeled antibodies emit continuous, exponentially decreasing, low-dose-rate radiation, whereas conventional external-beam radiotherapy delivers intermittent, fractional radiation at higher dose rates. It is one object of the present invention to provide an in vivo diagnostic apparatus utilizing radioimmunoassay or immunoradiometric assay for point-of-detection locating the source of the bodily abnormality.

Techniques for detecting primary association between antigen and antibody are outlined herein for reference, including equilibrium dialysis, fluorescence quenching or enhancement, Farr technique, gel permeation chromatography, ultracentrifugation, stopped-flow analysis, temperature-jump, isotopic dilution, radioimmunoassay, immunoradiometric assay and the like. They are all routinely performed on an in vitro basis. Radioimmunoassay (RIA) and immunoradiometric assay (IRMA) methods are capable of measuring the primary reaction between antigen and a single antibody. RIA is an important technique for quantifying minutely small amounts of biological substances such as enzymes, hormones, steroids, and vitamins in blood, urine, saliva or other body fluids. In RIA, the antigen is labeled with a radioactive isotope, whereas in IRMA, the antibody is the labeled species. As is well known in the chemical reaction of antigen (A_{g}) and antibody (A_{b}) to form a primary combination (A_{b}A_{g}) of antigen and antibody, the equilibrium constant for the overall reaction can be expressed as K=[A_{b}A_{g}]/{[A_{b}]×[A_{g}]}, where [A_{g}], [A_{b}] and [A_{b}A_{g}] are the concentrations of antigen, antibody, and antigen-antibody conjugate, respectively. For polyclonal antiserum, the average avidity of the antibody populations will determine the equilibrium constant K.

The two standard procedures for RIA are termed competitive and sequential. In a “Competitive RIA”, all reactants are mixed together simultaneously. Labeled antigen (A_{g}*) and unlabeled antigen (A_{g}) compete for binding to the antibody. In such a system, the avidity of the antibody for both the labeled and unlabeled antigen must be the same. Under these conditions, the probability of the antibody binding the labeled antigen is inversely proportional to the concentration of unlabeled antigen; hence counts are inversely proportional to unlabeled antigen concentration. Radiolabeling of antigen with an isotope can cause changes in reactivity with the antibody. Therefore, labeled and unlabeled antigens should always be evaluated when a competitive assay is used to assure that the antibody reacts equally with each form.

In a “Sequential RIA”, unlabeled antigen is first mixed with excess antibody and binding is allowed to achieve equilibrium. Labeled antigen is then added and allowed to equilibrate. After separation, the bound and free counts are determined. With this approach, a higher fraction of the unlabeled antigen can be bound by the antibody than in a competitive assay, especially at low antigen concentrations. Sequential assays can provide a two- to four-fold increase in sensitivity compared to a competitive assay when the rate for the forward binding reaction is much higher than the rate for the reverse reaction.

An in vitro method for RIA assay relies upon some physical method of separation of free from bound label. The methods may include electrophoresis, adsorption, ion exchange, gel filtration, double antibody precipitation, polymer precipitation, solvent or salt precipitation, protein A, biotin-avidin, solid phase antibodies and the like. Of particular interest to the in vivo diagnosis, some of the aforementioned methods involve precipitation of the bound antigen from the solution by using a protein precipitant such as ammonium sulfate, ethanol, dioxane, polypropylene glycol or the like. The bound antigen can also be precipitate immunologically by using a second antibody. For example, if the primary antibody is derived from rabbits, the second antibody can be an antiserum raised against rabbit γ-globulin in goats or sheep. This method has the advantage that it can be used for practically any assay. It has the disadvantage, however, that it usually requires longer assay times and additional steps. The measured bound antigen (A_{g}*) is compared to a typical RIA calibration curve that correlates the dose response percent bound versus concentration.

The IRMA has the advantage of not requiring a quantity of purified antigen because the antigen need not be labeled. This also obviates potential problems which may be caused by iodination of labile antigens. Antibodies are more stable proteins and are less difficult to label without damaging the protein's function. Typically, a “sandwich” or two-site IRMA method is used. In these systems, antibody is first attached to a solid phase by passive adsorption or by using reactions which result in a covalent binding of the antibody to the solid phase. Antigen from the sample is then allowed to react with the solid phase antibody, other protein is washed away, and a labeled antibody is added which reacts with the bound antigen through a second and distinct antigenic determinant. After washing again, the bound counts are determined and are directly proportional to the concentration of antigen. IRMA is a common approach for protein antigen measurement, due to its simplicity and ease of application in the routine laboratory.

Rubin and Strauss in U.S. Pat. No. Re. 35,152 disclose a method of detecting an inflammation site in an individual by administering to the individual a diagnostically effective amount of detectably labeled immunoglobulin. The method could detect the inflammation site only under the conditions when the labeled immunoglobulin could substantially accumulate at the inflamed site. This method has several disadvantages to detect and diagnose the body abnormality. The first drawback is its correct labeling of the immunoglobulin so as to accumulate substantially to be detectable isotopically. Extra labeled immunoglobulin not accumulated at the site pose side radiological effects to the patient. Secondly, to accurately place a radioactive detector next to the inflamed site is not trivial, since the whereabouts of the inflamed site is unknown in the first place. It is one object of the present invention to provide an in vivo diagnostic apparatus utilizing radioimmunoassay or immunoradiometric assay for point-of-detection locating the source of the bodily abnormality.

Agglutination Assay

Agglutination assays have been used for many years for the qualitative and quantitative measurement of antigens and antibodies. In an in vitro agglutination method, the visible clumping of particulates such as cells, bacteria, and latex particles is used as an indicator of the primary reaction of antigen and antibody. Agglutination methods require stable and uniform particulates, pure antigen and specific antibody. As with all immunochemical reactions in which aggregation is the measured endpoint, the ratio of antigen and antibody is critical. Extremes in antigen or antibody concentration will result in inhibition of aggregation.

In the case of a weak antigen-antibody reaction, enhancements may be achieved by lowering the ionic strength or introducing polymeric molecules such as polymerized albumin, dextran, polybrene, polyvinylpyrrolidone, or polyethylene glycol. The direct agglutination technique is used for the direct and indirect detection of antigens present on the surface of cells or particles. This technique is generally more sensitive than the precipitin reaction because the bulk of the cell provides added mass for visualization of the reaction. Specific agglutination is the clumping of bacteria, erythrocytes, and so forth in the presence of homologous antibody.

The classic agglutination test could be conducted either on a slide or in a test tube. Micro procedures that require very small quantity of costly reagents are being used with increasing frequency. The basic principles for agglutination test is well known to one skilled in the art and is fully taught in the Textbook of Clinical Chemistry (edited by N W Tietz, published by W. B. Saunders Company 1986). It is one embodiment of the present invention to provide an in vivo apparatus applying agglutination technique for detecting and locating the source of or site-specific markers in a patient's heart.

Nephelometry

Nephelometry is defined as the detection of light energy scattered or reflected toward a detector that is not in the direct path of the transmitted light. Common nephelometers measure scattered light at right angles to the incident light. The ideal nephelometric instrument would be free of stray light; neither light scatter nor any other signal would be seen by the detector when no particles are present in solution in front of the detector. However, due to stray light generating components in the optics path as well as in the sample curve or sample itself, a truly dark field situation is difficult to obtain when making nephelometric measurements. Some nephelometers are designed to measure scattered light at an angle other than 90° in order to take advantage of the increased forward-scatter intensity caused by light scattering from larger particles, e.g., immune complexes. Examples of analytes measured by nephelometry method include immunoglobulins (IgG, IgA, IgM, IgE), specific proteins (C3, C4, haptoglobin, transferrin, alpha₁-antigtrypsin, β-lipoproteins, fibronectin, human placental lactogen, albumin), coagulation factors (e.g., antithrombin III), therapeutic drugs (theophylline, gentamicin, phenytoin, phenobarbital), and the like.

When electromagnetic radiation, such as light, impinges upon a particle, its electrons become subject to a force in one direction and its nuclei to a force in the opposite direction, causing the electrons about the particle to oscillate in synchronism with the electric field of the incident light. The nephelometry is applicable for relatively clear solutions, where the transmission of light in the forward direction is greater than 95%, with some advantage in sensitivity when measuring low-level antigen-antibody reactions. The basic principles for nephelometry test is well known to one skilled in the art and is fully taught in the Textbook of Clinical Chemistry (edited by N W Tietz, published by W. B. Saunders Company 1986). It is one embodiment of the present invention to provide an in vivo apparatus applying nephelometry technique for detecting and locating the source of or site-specific markers in a patient's heart

B-Type Natriuretic Peptide (BNP) Assay

B-type natriuretic peptide (brain natriuretic peptide, BNP) and atrial natriuretic peptide (ANP) act as a dual system in regulating blood pressure and fluid balance. BNP was first identified in the porcine brain and subsequent studies have demonstrated that the heart is the major source of circulating BNP. BNP is stored in and secreted predominantly from membrane granules in the heart ventricles, and is continuously released from the heart in response to both ventricle volume expansion and pressure overload. It is one embodiment of the present invention to provide an in vivo apparatus for detecting and locating the source or rich site of BNP in a patient's heart. By locating the specific BNP secreting and/or storage site in a patient, certain types of abnormal bodily functions (for example, congestive heart failure) may be identified and/or therapeutically treated.

Physiological actions of BNP are mediated through a guanylate cyclase-linked receptor, natriuretic peptide receptor A (NPR-A). The NPR-A, found in various tissues, is a transmembrane protein composed of an extracellular binding site and an intracellular tail that catalyzes the conversion of guanosine triphosphate to cyclic guanosine monophosphate (cGMP). The cGMP, which has potent vasodilatory actions, acts as a second messenger of BNP. BNP is reportedly degraded through enzymatic cleavage by neutral endopeptidase, which opens the ring structure of BNP and inactivates the molecule.

ANP and BNP are activated by atrial and ventricular distension due to increased intra-cardiac pressure. These natriuretic peptides have both natriuretic and diuretic properties: they raise sodium and water excretion by increasing the glomerular filtration rate and inhibiting sodium reabsorption by the kidney. Congestive heart failure (CHF) occurs when the heart cannot deliver a sufficient blood supply to the body. Diagnosing CHF in very early stage permits early intervention that might prevent the disease from advancing. It is particularly important to identify and locating the specific BNP secreting site in vivo so that certain types of congestive heart failure may be determined.

Various studies have demonstrated that circulating BNP concentrations increase with the severity of CHF. BNP concentrations are normally much lower than ANP concentrations, but as the severity of CHF advances, plasma BNP increases progressively more than respective ANP values. Therefore, BNP appears to be a more useful marker to distinguish between normal subjects and patients in the early stages of CHF. There is reportedly a positive correlation between blood BNP concentrations and left ventricular end diastolic pressure and inverse correlation to left ventricular function (Kawai et al., American Heart Journal. 2001;141(6):925-932). However, the in vitro diagnosis as disclosed in prior art does not allow the in vivo identification of the potential heart problems using the BNP/ANP as markers.

An in vitro diagnostic device for the quantification of BNP in plasma and whole blood containing murine monoclonal and polyclonal antibodies against BNP, labeled with a fluorescent dye and immobilized on the solid phase and stabilizers can be obtained from Biosite Diagnostics, Inc. A product insert describes the intended use, summary and explanation of the test, and procedure for The Triage BNP Test kit (manufactured by Biosite Diagnostics, Inc., San Diego), entire contents of which are incorporated herein by reference. Buechler in U.S. Pat. No. 6,156,279 discloses an analytical apparatus suitable for in vitro determining the presence of a target ligand in a test sample for the controlled movement of reagents without membranes. However, Buechler does not disclose an in vivo point-of-detection method for a target ligand. It is one object of the present invention to provide an in vivo diagnostic apparatus for point-of-detection locating the source of BNP.

C-Reactive Protein Test

A substance present in the sera of acutely ill patients and able to bind the C-polysaccharide on the cell wall of Streptococcus pneumoniae was first described in 1930. In 1941, it was shown to be a protein which was given the name C-reactive protein (CRP). Characterization of the protein and production of reliable antisera have led to highly specific, sensitive, and reproducible in vitro quantitative methods.

CRP consists of five identical, nonglycosylated, polypeptide subunits noncovalently linked to form a disk-shaped cyclic polymer with a M.W. of 115,000-140,000. CRP is normally present in plasma at a mean concentration <800 μg/dL. CRP is an inflammatory marker, a substance that the body produces in response to inflammation. High levels of CRP in the blood mean the patient has inflammation somewhere in the body. Other tests are needed to detect the cause and location of the inflammation. It is one object of the present invention to provide an in vivo diagnostic apparatus for point-of-detection locating the source of bodily abnormality.

CRP is one inflammatory marker that has been found to be an indicator of heart health. CRP may also prove to be a potential marker for predicting coronary artery disease and stroke, which are closely associated with inflammation of the blood vessels. However, CRP is not normally present in the blood of a healthy patient. Other conditions, including diabetes, glucose intolerance and high pressure, might cause a mild increase of less than 5 micrograms per milliliter of blood. Perhaps the main role of CRP is to recognize potentially toxic autogenous substances released from damaged tissue, to bind them, and then to detoxify them or clear them from the blood. Yeh et al. (Circulation 2001;104:9754-975) and Chew et al. (Circulation 2001;104:992-997) reported that elevated baseline CRP levels before percutaneous coronary intervention are associated with a progressive increase in the risk of death or myocardial infarction at 30 days.

The C-reactive protein (CRP) test is a blood test that measures the level of CRP in the blood. CRP was frequently reported as a substance produced by the liver that increases when there is inflammation somewhere in the body. However, Kuta and Baum (J Exp Med 1986;164(1):321-326) reported that CRP is produced by a small number of normal human peripheral blood lymphocytes and confirms its first description of extrahepatic synthesis of CRP. Therefore, there is a clinical need for an in vivo catheter-based apparatus for measuring CRP at the site of inflammation; the point-of-detection site-specific CRP detection. Other marker resulting from inflammation may be a suitable alternative marker that can be identified by the in vivo diagnostic apparatus and method thereof of the present invention. The “point-of-detection” and “site-specific” are referred in the present invention as the origin or source of the marker or ligand of interest.

Impedance Test

As an illustration, a biosensor comprising an antibody-loaded substrate can be used to detect and quantify the antigen by measuring the impedance change of the biosensor. More particularly, the principles can be applied to detect the marker when the marker reacts with its counterpart antibody causing changes in impedance or other measurable parameters. Stetter et al. in U.S. Pat. No. 5,512,882 and U.S. Pat. No. 5,567,301 disclose an apparatus for the detection of a vapor of a selected chemical substance induces a sensor whose impedance changes upon exposure to such a vapor, the entire contents of which are incorporated herein by reference. Stetter et al. further characterizes the biosensor having at least one covalently bound antibody immobilized on a substrate material and impedance detection means for measuring an impedance of said sensor. However, Stetter does not disclose a miniaturized in vivo diagnostic apparatus for locating the source of a marker.

It is one object of the present invention to provide an in vivo diagnostic apparatus using impedance test methods for point-of-detection locating the source of bodily abnormality.

Apoptosis

Several research groups have demonstrated apoptotic cell death in atherosclerotic plaques. The significance of apoptosis in atherosclerosis depends on the stage of the plaque, localization and the cell types involved. Both macrophages and smooth muscle cells undergo apoptosis in atherosclerotic plaques. It is also reported that apoptosis of macrophages is mainly present in regions showing signs of DNA synthesis/repair while smooth muscle cell apoptosis is mainly present in less cellular regions and is not associated with DNA synthesis or repair. It is suggested that apoptosis is part of response-to-injury defense mechanism in atherosclerosis, however, its consequences on plaque biology may vary depending on the stage of plaque and other systemic or local factors. It is one object of the present invention to detect the markers associated with cells apoptosis in vivo leading to appropriate therapeutic treatments.

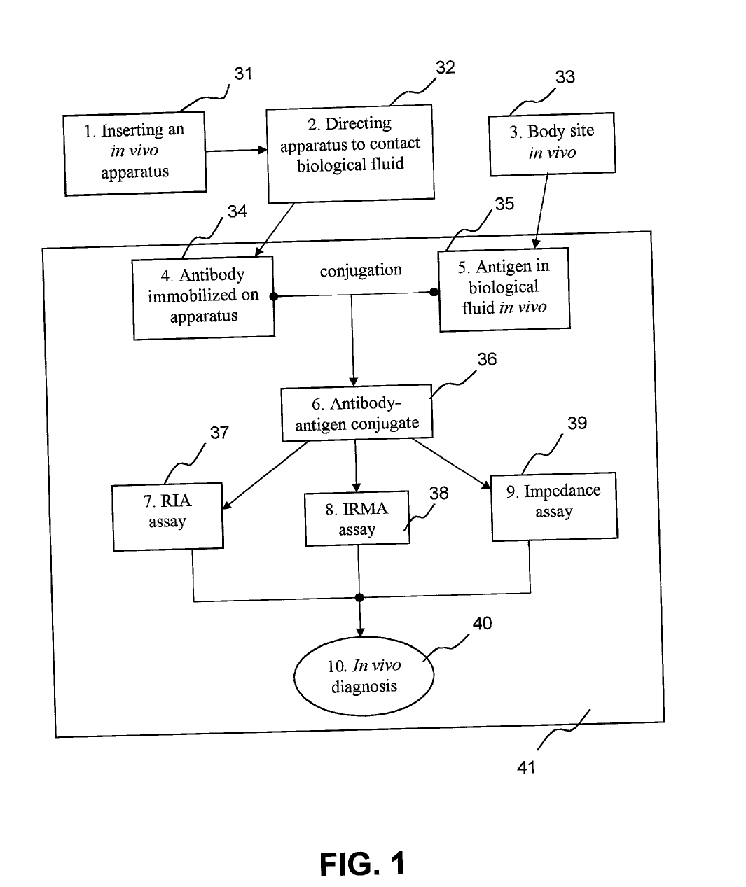

FIG. 1 shows a schematic diagram showing a method for diagnosing an antigen in vivo comprising a diagnosing element having antibody immobilized 34 on the diagnosing element that is conjugate-able to the antigen adapted to form antigen-antibody conjugate 36 for assay according to the principles of the present invention. A method for diagnosing an antigen in vivo may comprise 1) inserting an in vivo apparatus 31 into a body site 33 of a patient, wherein the body site 33 has antigen in the biological fluid in vivo 35; 2) introducing a diagnosing element to contact 32 with a biological fluid containing said antigen 35 at a site inside a patient, wherein the diagnosing element comprises antibody conjugate-able to said antigen; 3) quantifying the antigen-antibody conjugate by means for assaying said antibody in vivo by RIA assay 37, by IRMA assay 38 or Impedance assay 39; and/or 4) prorating to a reference quantity of antigen for the process of in vivo diagnosis 40 of the present invention. The in vivo portion 41 of the diagnosis method is illustrated in FIG. 1. The term “conjugate-able” is synonymous in this invention as “specific to” or “exhibiting affinity for”.

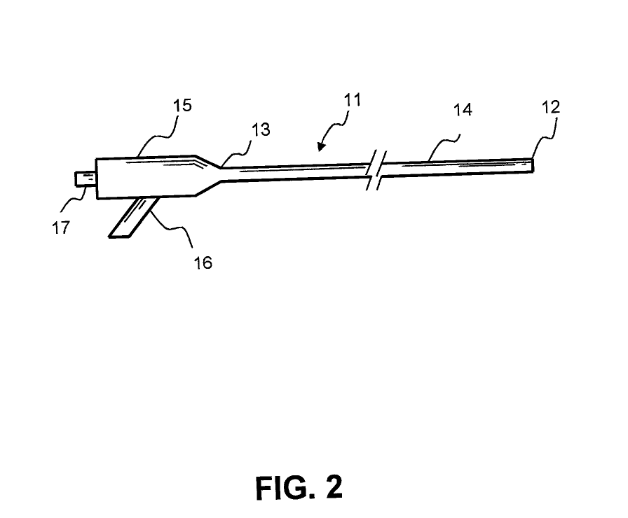

FIG. 2 shows a medical apparatus 11 having a diagnosing element mounted at about a tip portion 18 of the apparatus adapted for percutaneously inserting into a body conduit of a patient for antigen assay or detecting the markers. The apparatus 11 may comprise a shaft 14 having a shaft distal end 12, a shaft proximal end 13 and a lumen therebetween. The apparatus 11 or catheter may also comprise a holder 15 secured to the proximal end 13 of the catheter shaft 14. The holder 15 may have a plurality of connector elements 16, 17 for communicating the data or material between the apparatus 11 and the external instrument 26 or 46. For example, the connector element 16, or 17 may be used for relaying the impedance data from the tip section 18 to an external impedance measuring instrument 26. The diagnosing element for antigen assay in vivo of the present invention may be mounted at about a tip portion of a catheter adapted for percutaneously inserting into a body conduit of a patient. It is also applicable to be mounted at a tip portion of a trocar, a handpiece, a probe, an optic fiber, an endoscopic instrument, a guidewire, a cannula, and/or other suitable insertable biopsies devices or forceps.

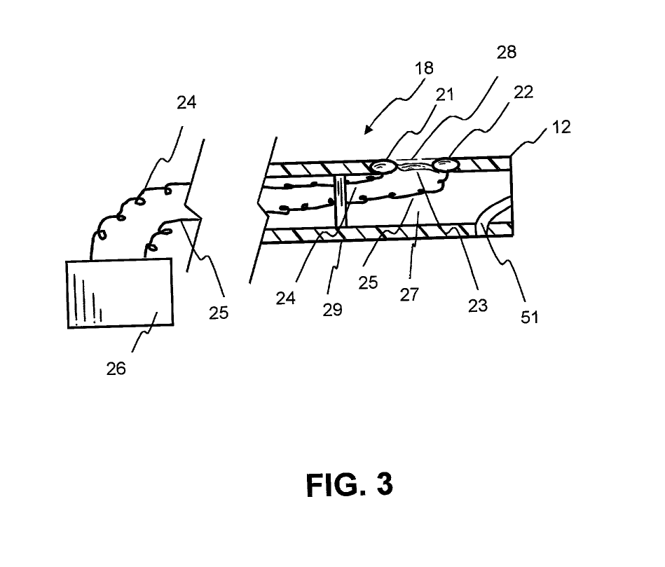

FIG. 3 shows one embodiment of the tip section 18 of the medical apparatus 11 of FIG. 2, having the capability of diagnosing an antigen in a patient in vivo. The apparatus may comprise an immobilized antibody mass 23 in a diagnostically effective amount of conjugating with the surrounding antigen in the biological fluid from a patient. The antibody mass 23 is placed and immobilized onto a pair of spaced-apart metal contact-electrodes 21, 22. There is an opening of the shaft 14 at about the tip section 18 for allowing biological fluid to contact the antibody 23. A one-way check valve 28 may be provided to allow the biological fluid to flow uni-directionally into the lumen 27 of the apparatus 11. The tip section 18 may also comprise a stopper 29 to confine the antigen-antibody reaction and diagnosis within the very tip portion of the apparatus 11. The quantification of the antigen-antibody conjugate is performed by means for assaying said antigen-antibody conjugate in vivo by impedance detection means for measuring an impedance between two spaced-apart metal contact-electrodes 21, 22 mounted on the diagnosing element, the antibody being coupled to both ends of said metal contact-electrodes that are mounted on the diagnosing element at about a tip portion of a catheter adapted for percutaneously inserting into a body conduit of a patient. The apparatus may also provide a distal guidewire channel 51 for riding the apparatus 11 onto a pre-inserted guidewire.

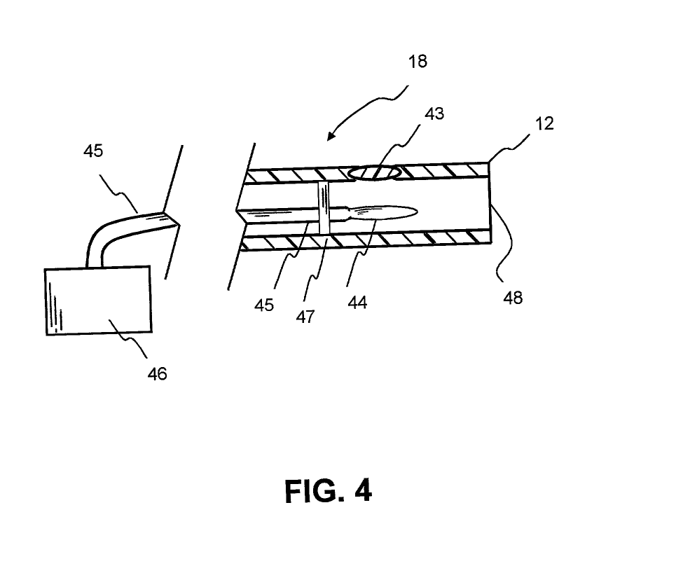

Alternatively as shown in FIG. 4, the connector element 17 may be used for inserting an inner catheter 45 within the lumen of the apparatus 11 to the tip section 18, wherein the inner catheter comprises a radioactivity detector/counter 44 at its tip portion for quantifying the antigen-antibody conjugate. One end of the inner catheter 45 may be coupled to an external instrument 46 supplying the radioactivity detecting/counting capability. The distal end 12 of the apparatus 11 may have a sealed wall 48 to avoid contamination of the lumen 27 by surrounding biological fluid. The immobilized antibody 43 is conjugate-able to antigen in the surrounding biological fluid of a patient in vivo. The tip section 18 may also comprise a stopper 47 to confine the antigen-antibody reaction and fluid diagnosis within the very tip portion of the apparatus 11.

The apparatus and method of in vivo diagnosing a marker may be applicable to other cardiovascular markers, such as troponins (T, C, and I). The troponins are a group of intimately related regulatory proteins located in striated muscle. In acute myocardial injury, there is a biphasic release of troponins T and I into the serum. Troponin in the cell, presumed to originate from the cytoplasm, is released 3 to 5 hours after loss of membrane function. A late phase of continued release of troponin for 5 days or more follows and is thought to be associated with destruction of the contractile apparatus and cell death. Troponin I is solely confined to the myocardium and has been shown to be a highly specific marker for the detection of myocardial injury. Typically, troponin I is measured in systemic venous samples for a period of 48 hours postoperatively and also immediately perioperatively in blood samples obtained directly from a cannula positioned in the coronary sinus. It is one object of the present invention to provide an in vivo diagnostic apparatus using suitable assay or test methods for point-of-detection locating the source of myocardial abnormality.

From the foregoing description, it should now be appreciated that a novel and unobvious diagnostic apparatus for diagnosing an antigen in vivo has been disclosed. While the invention has been described with reference to a specific embodiment, the description is illustrative of the invention and is not to be construed as limiting the invention. Various modifications and applications may occur to those who are skilled in the art, without departing from the true spirit and scope of the invention.

What is claimed is:

1. A method for diagnosing an antigen in vivo comprising:
introducing a diagnosing element to contact with a biological fluid containing said antigen at a site inside a patient, wherein said diagnosing element comprises antibody conjugate-able to said antigen adapted to form an antigen-antibody conjugate; and
quantifying the antigen-antibody conjugate, wherein said quantification of the antigen-antibody conjugate is performed by means for assaying said antigen-antibody conjugate in vivo.

2. The method of claim 1 further comprising a step of prorating a quantity of the antigen-antibody conjugate to a reference quantity of antigen.

3. The method of claim 1, wherein said means for assaying said antigen-antibody conjugate is either an immunoradiometric assay or a radioimmunoassay.

4. The method of claim 1, wherein said antigen is C-reactive protein.

5. The method of claim 1, wherein said antigen is B-type natriuretic peptide.

6. The method of claim 1, wherein said antigen is interlukin-6.

7. The method of claim 1, wherein said diagnosing element is mounted at about a tip portion of a catheter adapted for percutaneously inserting into a body conduit of a patient.

8. The method of claim 1, wherein said biological fluid is selected from the group consisting of whole blood, plasma, serum, urine, and saliva.

9. The method of claim 1, wherein said biological fluid is selected from the group consisting of cerebrospinal fluid, peritoneal fluid, pleural fluid, lymphatic fluid, and joint fluid.

10. The method of claim 1, wherein said quantification of the antigen-antibody conjugate is performed by means for assaying said antigen-antibody conjugate in vivo by impedance detection means for measuring an impedance between two spaced-apart metal contact-electrodes mounted on the diagnosing element, the antibody being coupled to both ends of said metal contact-electrodes that are mounted on the diagnosing element at about a tip portion of a catheter adapted for percutaneously inserting into a body conduit of a patient.

11. A method for assessing vulnerability of a patient in vivo having vulnerable plaque comprising:
introducing a diagnosing element to contact with a body fluid of the patient at a site inside the patient, the body fluid containing a substance associated with the vulnerable plaque, wherein said diagnosing element comprises a receptor protein conjugate-able to said substance;
quantifying the receptor protein that conjugate-ably reacts with said substance; and
comparing a level of the reacted substance of the patient to a reference data pool from healthy patients.

12. The method of claim 11, wherein the substance is selected from a group consisting of C-reactive protein, interlukin-6, B-type natriuretic peptide, troponin T, troponin C, troponin I, and tissue-type plasminogen activator (t-PA) antigen.

13. The method of claim 11, wherein the method for quantifying the receptor protein that conjugate-ably reacts with said substance is accomplished in vivo.

14. The method of claim 11, wherein the method for quantifying the receptor protein that conjugate-ably reacts with said substance comprises radioimmunoassay.

15. The method of claim 11, where the method for quantifying the receptor protein that conjugate-ably reacts with said substance comprises an agglutination procedure.

16. The method of claim 11, where the method for quantifying the receptor protein that conjugate-ably reacts with said substance comprises a nephelometry procedure.

17. A method for assessing an individual's risk profile of developing a future cardiovascular disorder with atherosclerotic disease comprising:
obtaining at least a level of a marker of inflammation about a region of atherosclerotic disease in vivo in the individual;
comparing the at least a level of the marker with levels from various sites of said region of the atherosclerotic disease; and
identifying a site inside the individual having the level of the marker sufficiently higher than the remaining levels adapted for therapeutic treatment.

18. The method of claim 17, wherein the marker is C-reactive protein.

19. The method of claim 17, wherein the marker is interlukin-6.

20. The method of claim 17, wherein the marker is tissue-type plasminogen activator (t-PA) antigen, a troponin, or B-type natriuretic peptide.
